(19) United States
(12) Patent Application Publication
(10) Pub. No.: US 2013/0030260 A1
(43) Pub. Date: Jan. 31, 2013

(54) SYSTEM AND METHOD FOR BIOMETRIC HEALTH RISK ASSESSMENT

(71) Applicant: Sean Hale, Muncie, IN (US)

(72) Inventor: Sean Hale, Muncie, IN (US)

(21) Appl. No.: 13/559,759

(22) Filed: Jul. 27, 2012

Related U.S. Application Data
(60) Provisional application No. 61/512,602, filed on Jul. 28, 2011.

(51) Int. Cl. A61B 5/0205; A61B 5/145

(52) U.S. Cl. 600/301

(57) ABSTRACT

A system and method for generating and displaying a health risk assessment report to a patient in real time is provided. The system receives a plurality of biometric data values for a patient and automatically processes subsets of the data to determine at least one category grade point average or letter grade. An overall health risk grade point average or letter grade is determined and presented to the patient along with the individual category grades. A simulated category letter grade and overall letter grade may also be presented which reflect selected adjustments to at least one of the biometric data values. The biometric data values may be based on real time measurement of a biosample taken from the patient in addition to manually entered data.

### CROSS REFERENCE TO RELATED APPLICATIONS

This application claims the benefit of U.S. Provisional Patent Application No. 61/512,602 filed Jul. 28, 2011, which is hereby incorporated by reference in its entirety.

### TECHNICAL FIELD

The present invention relates generally to medical health information systems and, more particularly, to a system and method for biometric health risk assessment.

### BACKGROUND

Various systems for providing health or wellness information to patients are known in the art. Such systems typically analyze input biometric data or biosamples taken from a patient in order to produce a wellness report. Unfortunately, these reports often display the data in a format which is overly complicated and confusing to the patient. The reports may even require the assistance of a medical doctor to interpret the results and explain them to the patient. Even after hearing or viewing the results, patients may still be unclear as to what steps they should take to improve their overall health with respect to the data listed in the report. There is therefore a need for improved systems which provide an accurate and complete health risk assessment report, while still presenting the data in a format which is easily understood by patients.

### SUMMARY

According to one aspect of the present disclosure, a method for generating and displaying a health risk assessment report to a patient in real time is presented, including: receiving a plurality of biometric data values for a patient; automatically processing a first subset of the plurality of biometric data values to determine a first category health risk score for the patient in a first health risk category; converting the first category health risk score to a first category health risk letter grade; automatically processing a second subset of the plurality of biometric data values to determine a second category health risk score for the patient in a second health risk category; converting the second category health risk score to a second category health risk letter grade; determining an overall health risk letter grade for the patient based on at least the first category health risk score and the second category health risk score; and displaying the first category health risk letter grade, the second category health risk letter grade, and the overall health risk letter grade to the patient in real time using an electronic display. A simulated first category health risk letter grade and a simulated overall health risk letter grade may be presented to the patient in real time using the electronic display, wherein the simulated first category health risk letter grade and the simulated overall health risk letter grade reflect a selected adjustment to at least one of the first subset of the biometric data values. The method is preferably performed using a personal computing device having a processor and memory configured to perform the method steps.

According to another aspect, a system for generating and displaying a health risk assessment report to a patient in real time, comprising: a personal computing device having a memory and a processor, the memory comprising computer-readable instructions which cause the personal computing device to receive a plurality of biometric data values for a patient; and an electronic display operably connected to the personal computing device. The memory comprises computer-readable instructions which cause the personal computing device, using the processor, to automatically process a first subset of the plurality of biometric data values to determine a first category health risk score for the patient in a first health risk category and convert the first category health risk score to a first category health risk letter grade. The memory also comprises computer-readable instructions which cause the personal computing device, using the processor, to automatically process a second subset of the plurality of biometric data values to determine a second category health risk score for the patient in a second health risk category and convert the second category health risk score to a second category health risk letter grade. The memory also comprises computer-readable instructions which cause the personal computing device, using the processor, to determine an overall health risk letter grade for the patient based on at least the first category health risk score and the second category health risk score. The memory also comprises computer-readable instructions which cause the personal computing device, using the processor, to present the first category health risk letter grade, the second category health risk letter grade, and the overall health risk letter grade to the patient in real time using the electronic display. Further forms, objects, features, aspects, benefits, advantages, and embodiments of the present invention will become apparent from a detailed description and drawings provided herewith.

### BRIEF DESCRIPTION OF THE DRAWINGS

FIG. 1 is schematic block diagram of a system for providing a biometric health risk assessment according to one embodiment of the present disclosure.

FIG. 2a is a first portion of a sample report generated by the system of FIG. 1.

FIG. 2b is a second portion of a sample report generated by the system of FIG. 1.

FIG. 3 is the report of FIG. 2a showing a blood pressure adjustment tool for creating a temporary comparison report.

FIG. 4 is the report of FIG. 2a showing a cholesterol adjustment tool for creating a temporary comparison report.

FIG. 5 is the report of FIG. 2a showing a weight management adjustment tool for creating a temporary comparison report.

FIG. 6 is the report of FIG. 2a showing a diabetes adjustment tool for creating a temporary comparison report.

FIG. 7a is a first portion of an aggregate health risk assessment report according to one embodiment.

FIG. 7b is a second portion of an aggregate health risk assessment report according to one embodiment.

FIG. 8 is a process flow diagram of a method for providing a health risk assessment report according to one embodiment.

### BRIEF DESCRIPTION OF THE PREFERRED EMBODIMENTS

For the purposes of promoting an understanding of the principles, reference will now be made to the embodiments illustrated herein and specific language will be used to describe the same. It will nevertheless be understood that no limitation of the scope of the invention is thereby intended. Any alterations and further modifications in the described processes, systems or devices, any further applications of the principles of the invention as described herein, are contemplated as would normally occur to one skilled in the art to which the invention relates, now and/or in the future.

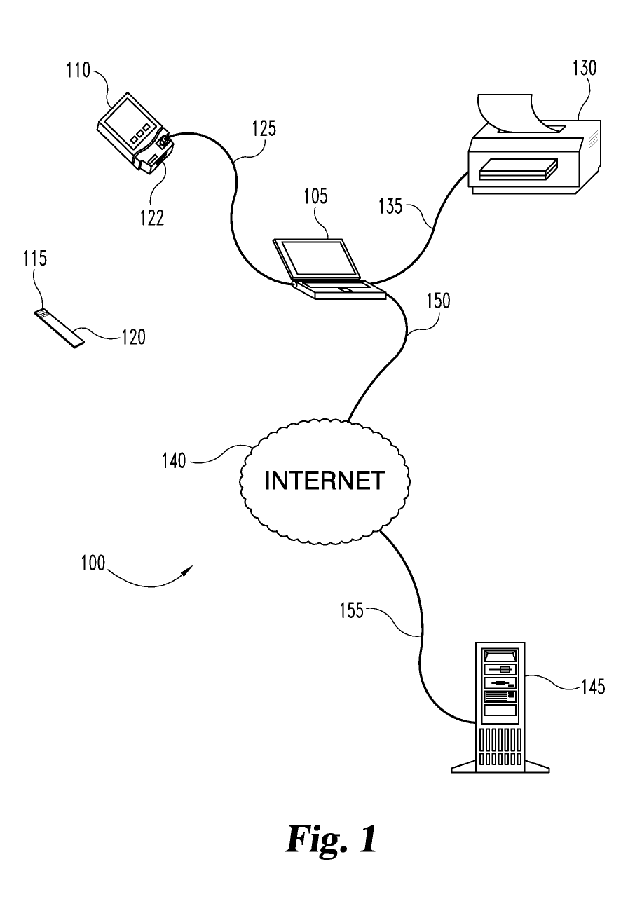

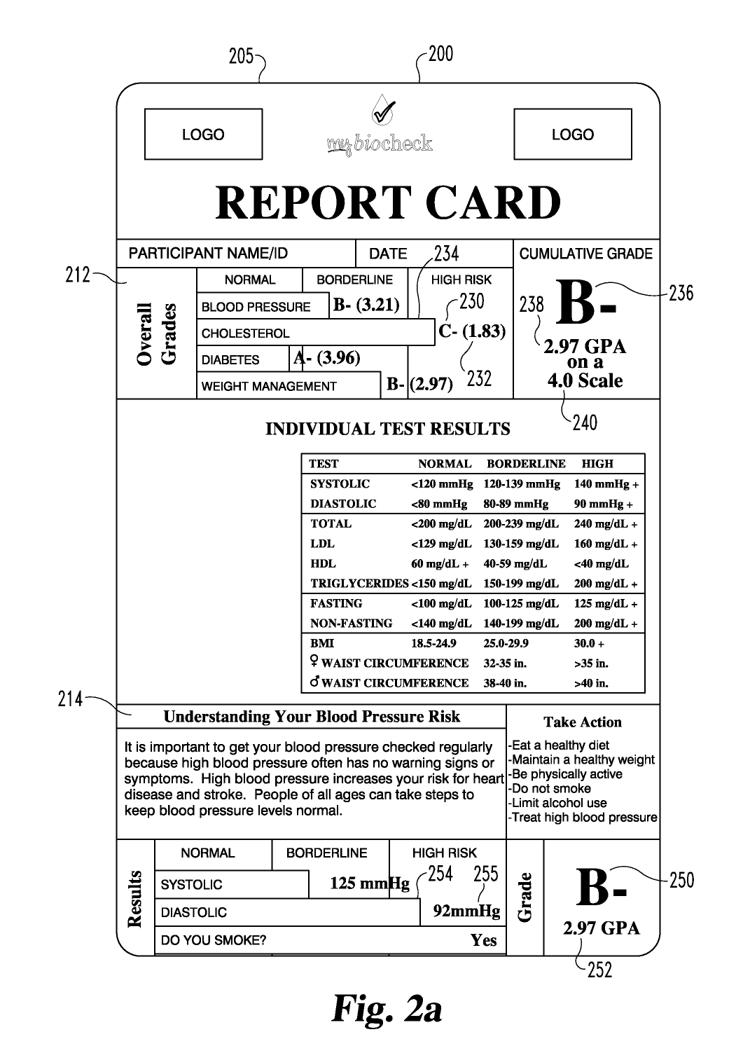

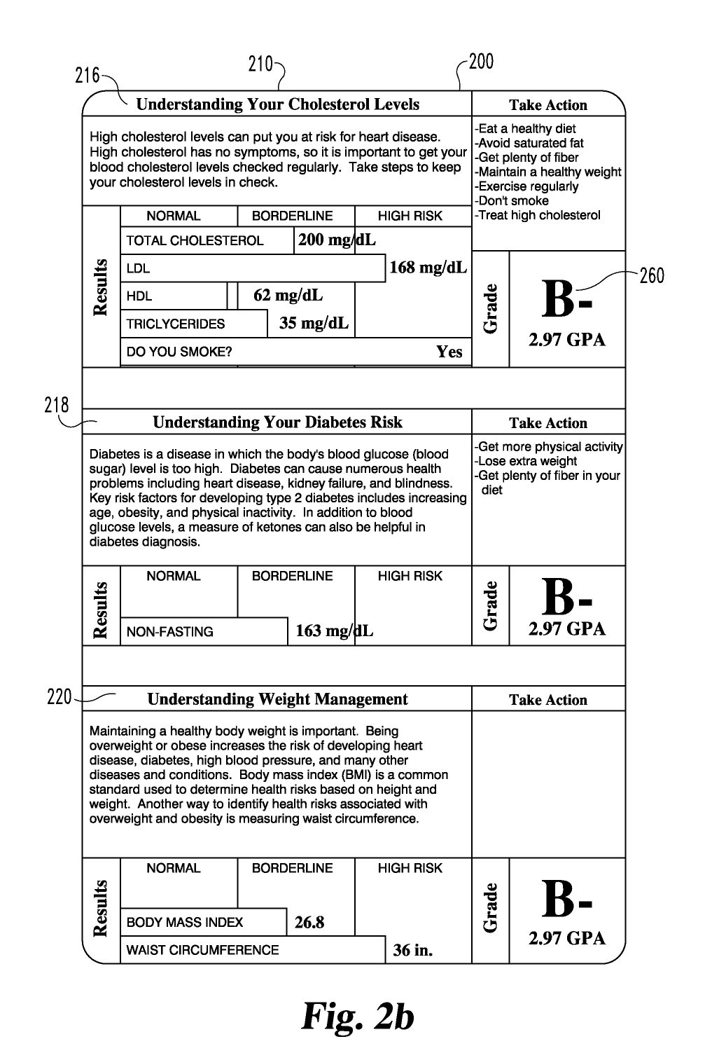

For the convenience of the reader, it should be initially noted that a drawing in which an element is first introduced is typically indicated by the left-most digit(s) in the corresponding reference number. For example, a component identified with a one-hundred series reference number (e.g., 100, 101, 102, 103, etc.) will usually be first discussed with reference to FIG. 1, and a component identified with a two-hundred series reference number (e.g., 200, 201, 202, 203, etc.) will usually be first discussed with reference to FIG. 2.

The system and method for biometric health risk assessment described herein shall be illustrated as implemented via computer software and hardware, with appropriate components and devices. Data collected by the various individual computers and devices may be centrally collected by a wired or wireless network (either public or private) if desired, subject to data redundancy protocols and patient privacy requirements and concerns.

FIG. 1 shows a diagrammatic view of a system 100 for generating and displaying a biometric health risk assessment report to a patient in real time according to one embodiment of the present disclosure. As illustrated, the system 100 may include a personal computing device 105, a diagnostic device 110, printer 130, and remote server 145. The personal computing device 105 is operatively connected to the diagnostic device 110 via cable 125 and to printer 130 via cable 135. The personal computing device 105 may be further connected to remote server 145 via network 140 using cables 150 and 155 or using wireless connections. As one non-limiting example, the system 100 may be used at a health screening fair or other event designed to promote public health and wellness. However, the system 100 may also be implemented in other contexts, including home environments, nursing homes, workplaces, or any location where patients wish to receive health risk assessment information.

In operation, the personal computing device 105 is programmed with computer-readable instructions for providing a graphical user interface for receiving biometric data values regarding a patient, such as height, weight, eating habits, etc. The data values may be input manually using a keyboard, touchpad, and/or touchscreen within or connected to the personal computing device 105. Additional data may be transmitted to the personal computing device 105 by the diagnostic device 110, such as information based on the analysis of a biosample 115 (e.g., blood) placed onto a test strip 120 and inserted into diagnostic port 122 of the device 110. As will be explained in further detail below, a health risk assessment report is generated by the system 100 and displayed for the patient on an electronic display incorporated within or connected to the personal computing device 105. The report is formatted as a “report card” with overall and individual category letter grades and grade point averages. Health information and reports for multiple patients may also be aggregated and optionally sent to remote server 145 for storage and later retrieval.

The personal computing device 105 may comprise any electronic digital computer known in the art having a processor and memory, electronic display, and an input device, such as a keyboard, mouse and/or touchpad. For example, the personal computing device 105 may comprise a laptop computer, a desktop computer, tablet computer or a handheld mobile computing device (e.g., iPhone, iPad, Blackberry, etc.). It shall be understood that the personal computing device 105 may communicate with the various components in the system 100 using wired or wireless mediums and formats.

The diagnostic device 110 may comprise any type of electronic device for receiving and analyzing a biosample 115 received from a patient. For example, the diagnostic device 110 may comprise a blood glucose meter which analyzes a drop of the patient's blood to determine the glucose level present in the blood. The blood is placed onto the test strip 120 and the test strip is inserted into the meter via port 122 and analyzed. In other embodiments, the diagnostic device 110 may comprise a cholesterol meter, which analyzes blood in a similar fashion to determine the patient's cholesterol level. Still other types of diagnostic devices may be connected to the personal computing device 105 such as urine analysis devices, electronic scales (for weight measurement), blood pressure monitoring devices, electro-cardiogram (EKG) machines, temperature measurement devices, and the like.

The remote server 145 may comprise any type of electronic digital computing device having a processor and memory, similar to personal computer device 105 described above. Each of the personal computing device 105 and remote server 145 may utilize any processor and memory known in the art. For example, the processors may be of the electronic variety defining digital circuitry, analog circuitry, or both. In one embodiment, each processor is of a conventional, integrated circuit microprocessor arrangement, such as one or more CORE™ processors (including CORE 2 Duo, Core i3, Core i7 and the like) or PENTIUM 4® processors supplied by INTEL Corporation of 2200 Mission College Boulevard, Santa Clara, Calif. 95052, USA. It shall be appreciated that other processors manufactured by INTEL or other suppliers would be suitable for use with the system and method described herein.

Each memory of the personal computing device 105 and remote server 145 may include one or more types of solid-state electronic memory, magnetic memory, or optical memory, just to name a few. By way of non-limiting example, each memory may include solid-state electronic Random Access Memory (RAM), Sequentially Accessible Memory (SAM) (such as the First-In, First-Out (FIFO) variety or the Last-In-First-Out (LIFO) variety), Programmable Read Only Memory (PROM), Electronically Programmable Read Only Memory (EPROM), or Electrically Erasable Programmable Read Only Memory (EEPROM); an optical disc memory (such as a DVD or CD ROM); a magnetically encoded hard disc, floppy disc, tape, or cartridge media; or a combination of any of these memory types. Also, each memory may be volatile, nonvolatile, or a hybrid combination of volatile and nonvolatile varieties.

Computer network 140 can be in the form of a wireless or wired Local Area Network (LAN), Virtual Private Network (VPN), the internet, a combination of these, or other network arrangement as would occur to those skilled in the art. The operating logic of system 100 can be embodied in signals transmitted over network 140, in programming instructions, dedicated hardware, or a combination of these.

FIGS. 2a and 2b collectively illustrate a sample screenshot of a health risk assessment report 200 generated by the system 100 according to one embodiment of the present disclosure. The report 200 may be presented as a graphical user interface, with selectable controls as will be explained further below. For clarity and ease of illustration, the report 200 is shown here in two pages (portions 205 and 210), although it should be understood that the portions 205 and 210 may be displayed to the user on a single screen view, depending on the size of the electronic display in the personal computing device 105 or according to user preference. The report 200 may include various sections which display health risk or wellness information in a variety of categories (sections 214, 216, 218 and 220), in addition to a combined overall results section 212.

The system 100 may provide report information for any category relating to health or wellness. In the illustrated embodiment, section 214 includes a display of the biometric data values relating to blood pressure which have been received for the patient. The data values may be entered by the patient or by a health care professional assisting the patient. As shown, the data values may include systolic blood pressure, diastolic blood pressure, and smoking status. The data values may be visualized using numerical indicators 255 and corresponding graphical indicators 254 (e.g., a bar graph) to indicate the relative risk level (normal, borderline, or high) of the individual data values 255. The system 100 uses the individual biometric data values for the category to determine a category risk score for the patient. To provide a more intuitive display, the category score is then converted to a letter grade (indicator 250) in the range of A to F, with optional pluses and minuses, and grade point average (indicator 252) in the range of zero to 4.0. Biometric data and corresponding letter grades and grade point averages for additional categories may also be displayed. For example, section 216 includes biometric data values relating to the cholesterol category, such as total cholesterol, low-density lipoprotein (LDL), high-density lipoprotein (HDL), triglycerides, and smoking status. Section 218 includes biometric data values relating to the diabetes category, such fasting status. Section 220 includes biometric data values for body mass index and waist circumference. Again, the biometric data values for each category are used to determine a category score, which is converted to a letter grade and grade point average for the respective category.

The individual category scores may be combined to determine an overall or cumulative letter grade and grade point average based on a given scale, shown here using indicators 236, 238 and 240 respectively. In one embodiment, the individual category scores are added equally and averaged to determine the overall letter grade and grade point average. In other embodiments, the category scores may be given differing weight when performing the calculation. Section 212 may also list an overall summary of the individual category letter grades (indicators 230) and grade point averages (indicators 232), with graphical indicators 234 optionally provided for ease of interpretation.

Once the user is presented with the report 200 containing information based on the actual or measured input biometric data values, she may wish to learn more about how changes in particular values may affect her overall wellness grade or grade point average. In certain embodiments, the system 100 allows the patient to select an appropriate section of the report (e.g., by clicking using a mouse or touching a touchscreen), which will activate an adjustment tool.

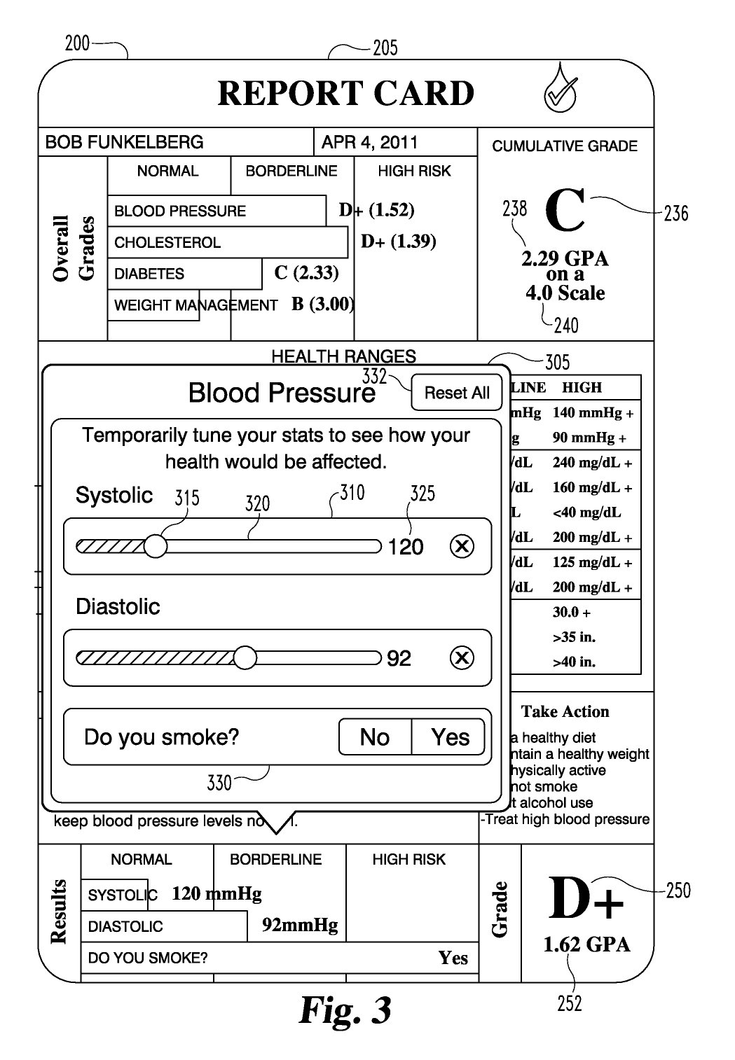

FIG. 3 illustrates an adjustment tool 305 for simulating changes to biometric data values relating to blood pressure. The adjustment tool 305 may include various graphical controls for adjusting the biometric values, such as slider 310 or question/answer box 330. To adjust the slider 310, and corresponding numerical indicator 325, the patient drags control button 315 to the left or right within the bar 320. As the patient adjusts the selected biometric data value, the displayed overall and category grades (indicators 236, 238, 250 and 252) will also change. This allows the patient to see how the adjusted value will affect their overall health. Reset button 332 may also be provided which allows the patient to reset the values back to their actual measured levels.

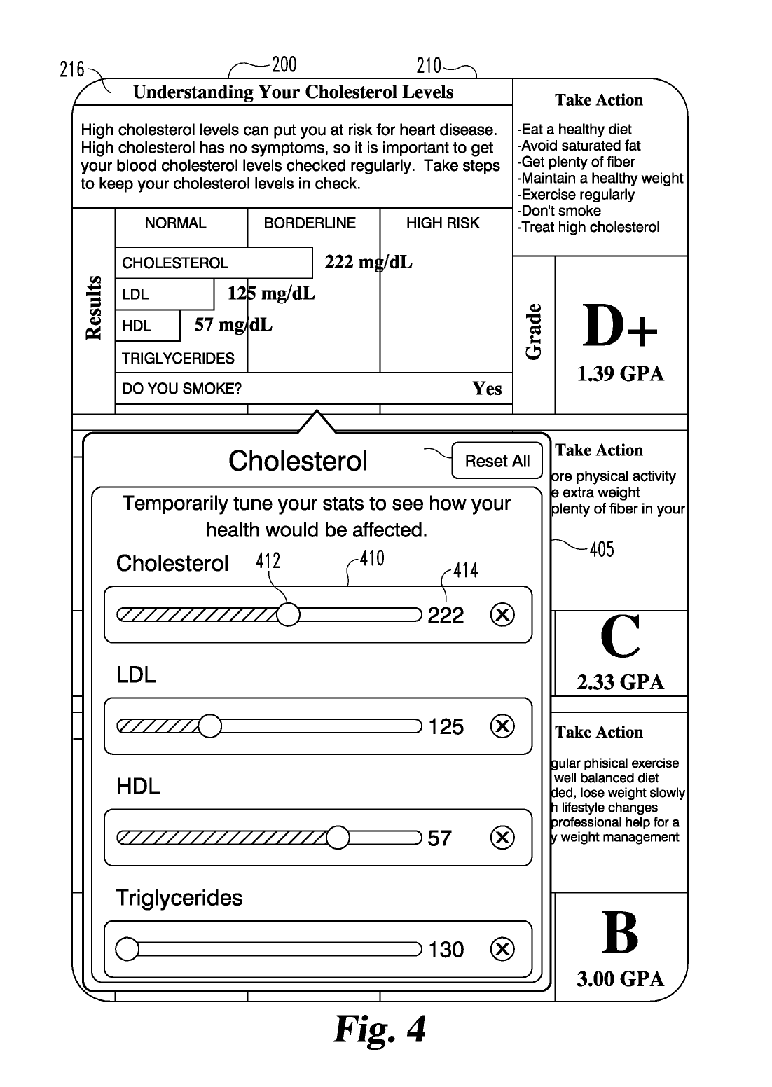

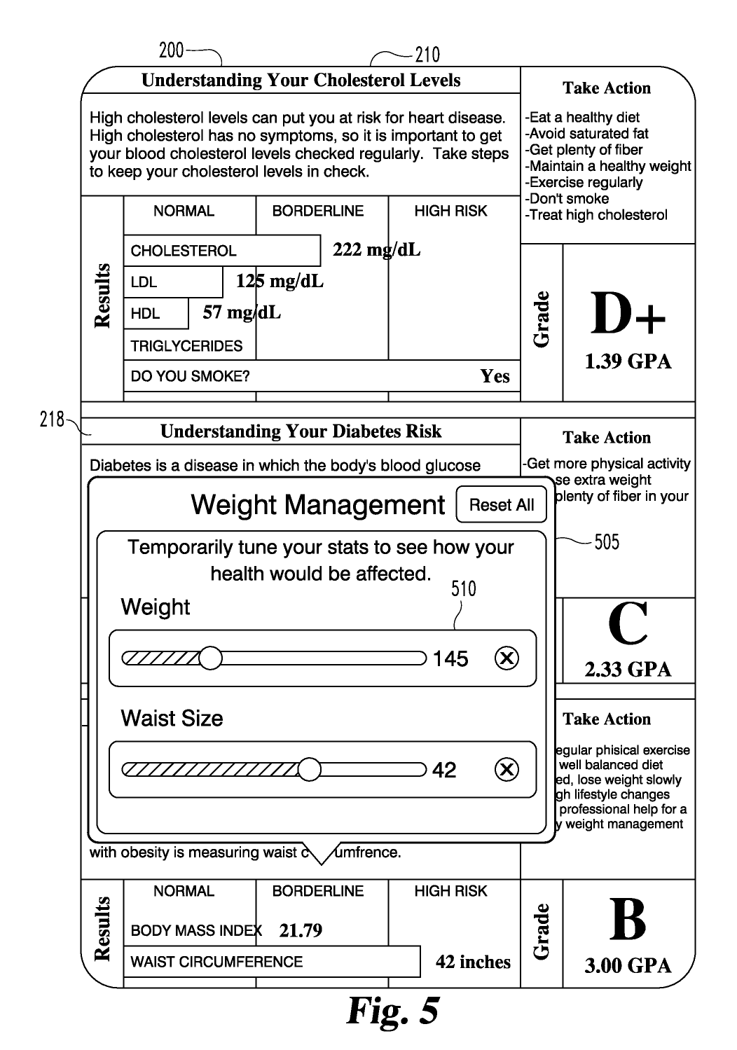

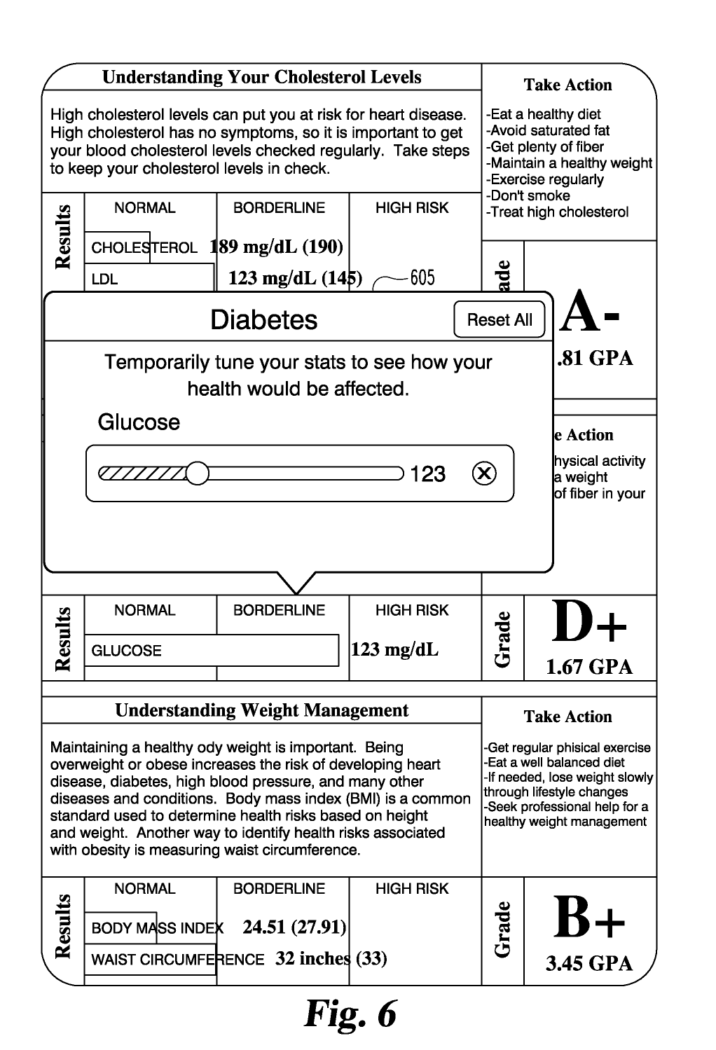

FIGS. 4-6 show additional adjustment tools for the other wellness categories. As shown in FIG. 4, an adjustment tool 405 may be provided for adjusting values related to cholesterol. Adjustment tool 405 includes sliders 410 which function similar to adjustment tool 305, using control buttons 412 and numerical indicators 414. FIG. 5 shows an adjustment tool 505 having controls 510 for adjusting values relating to weight management. FIG. 6 shows an adjustment tool 605 for adjusting values relating to diabetes risk. It shall be understood that in addition to sliders and question/answer boxes, other types of interface controls may be provided for manipulating or adjusting the various biometric data values.

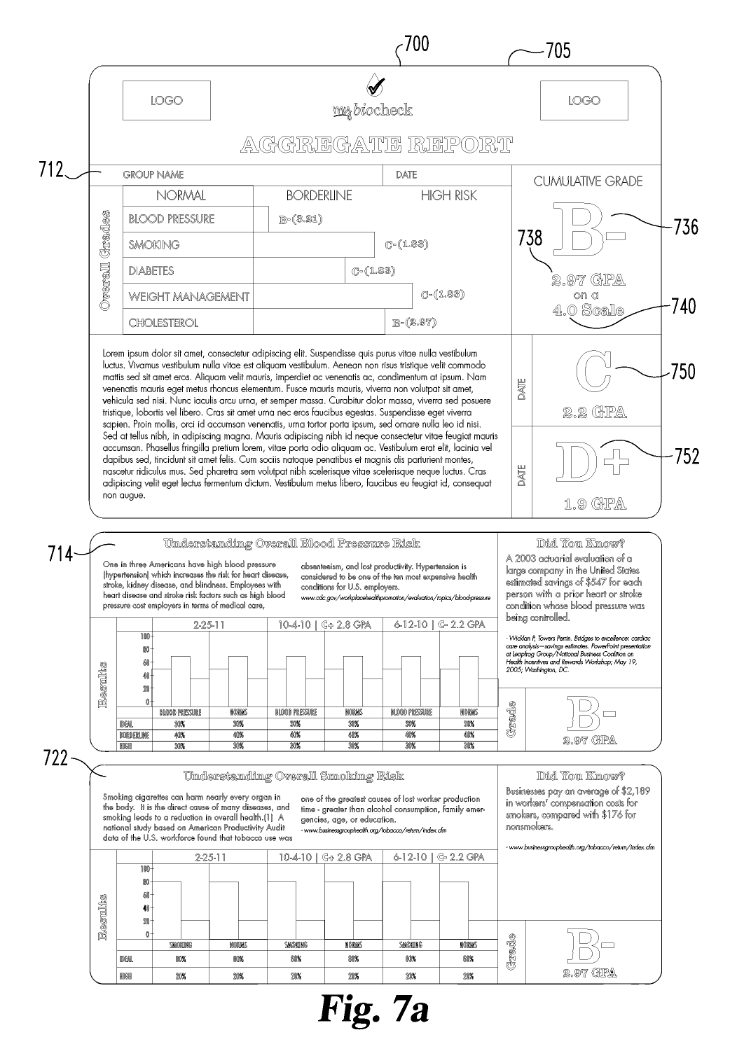

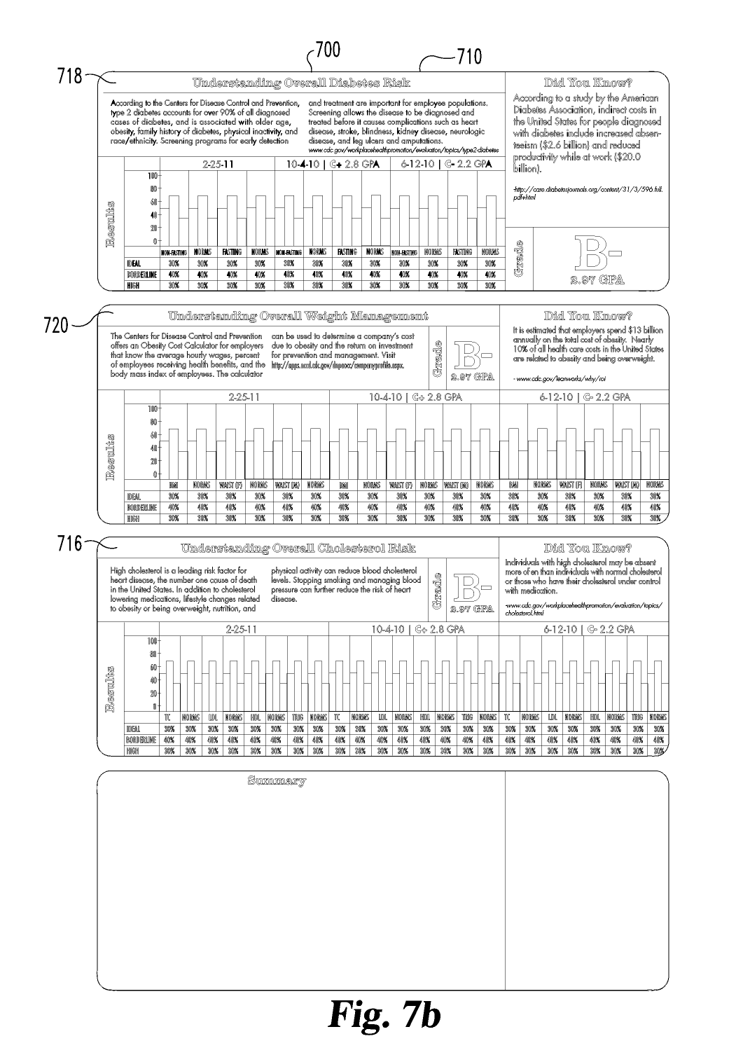

In certain embodiments, the scores, letter grades, and grade point averages for multiple patients may be collected, stored, and presented in aggregate form for use by an employer or other sponsoring entity, subject to permission of the patients and in compliance with the appropriate privacy concerns and government regulations. FIGS. 7a and 7b respectively illustrate portions 705 and 710 of an aggregate health risk assessment report 700 for a studied population. As shown, the report 700 includes an overall summary section 712, which lists the cumulative overall letter grade, grade point average, and scale (indicators 736, 738 and 740, respectively) for the population. In certain embodiments, indicators 750 and 752 may be provided which list letter grades from previous assessment dates for comparison to the current report. The aggregate report 700 may also include summaries of the individual category data for the population, such as blood pressure (section 714), diabetes (section 718), weight management (section 720), cholesterol (section 716) and smoking (section 722). The aggregated data may be stored on the personal computing device 105 or transmitted to the remote server 145 for storage and later retrieval.

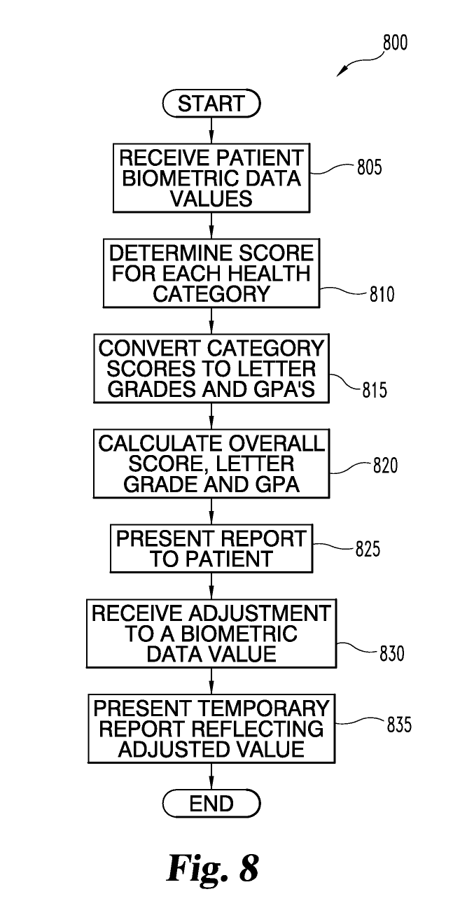

FIG. 8 illustrates a process 800 for generating and displaying a health risk or wellness assessment according to one embodiment of the present disclosure. The process begins at step 805 where the personal computing device 105 receives biometric data related to a patient. As described above for one embodiment, the data may comprise manually entered data based on a verbal or electronically administered questionnaire, and may further include the results of analysis of a biosample taken from the patient previously or at that time.

Once the biometric data has been received, the individual scores are determined for each health risk or wellness category (step 810). The scores are then converted into letter grades and grade point averages (step 815). At step 820, the individual scores are combined to produce an overall score, which is also converted to an overall letter grade and grade point average. The compiled data is then presented to the user in the form of a graphical user interface and report on the display of the personal computing device 105. The report may optionally be printed for the patient using printer 130 or provided in electronic form.

At step 830, adjustments to individual biometric data values may be received via the adjustment tools within the report interface. Based on the changes, a temporary or simulated report is generated and displayed which reflects the adjusted values (step 835). The simulated report may be saved and/or printed for future reference, along with the original report.

While the invention has been illustrated and described in detail in the drawings and foregoing description, the same is to be considered as illustrative and not restrictive in character, it being understood that only the preferred embodiment has been shown and described and that all changes, equivalents, and modifications that come within the spirit of the inventions defined by following claims are desired to be protected. The articles “the”, “a” and “an” are not necessarily limited to mean only one, but rather are inclusive and open ended so as to include, optionally, multiple such elements.

What is claimed is:

1. A method for generating and displaying a health risk assessment report to a patient in real time using a personal computing device, the personal computing device having a processor and memory configured to perform the method, comprising:
receiving a plurality of biometric data values for a patient;
automatically processing a first subset of the plurality of biometric data values to determine a first category health risk score for the patient in a first health risk category;
converting the first category health risk score to a first category health risk letter grade;
automatically processing a second subset of the plurality of biometric data values to determine a second category health risk score for the patient in a second health risk category;
converting the second category health risk score to a second category health risk letter grade;
determining an overall health risk letter grade for the patient based on at least the first category health risk score and the second category health risk score; and
displaying the first category health risk letter grade, the second category health risk letter grade, and the overall health risk letter grade for the patient in real time using at least one of an electronic display and a printer.

2. The method according to claim 1, further comprising:
presenting a simulated first category health risk letter grade and a simulated overall health risk letter grade to the patient in real time using at least one of the electronic display and the printer;
wherein the simulated first category health risk letter grade and the simulated overall health risk letter grade reflect a selected adjustment to at least one of the first subset of the biometric data values.

3. The method according to claim 1,
wherein at least one of said plurality of biometric data values is based on a measurement performed on a biosample taken from the patient at the same location where the health risk assessment report is being viewed by the patient; and
wherein the said measurement is performed using a diagnostic device.

4. The method according to claim 3, wherein said diagnostic device comprises a cholesterol meter.

5. The method according to claim 3, wherein said diagnostic device comprises a blood glucose meter.

6. The method according to claim 3,
wherein the biosample comprises blood.

7. The method according to claim 1,
wherein at least one of said plurality of biometric data values comprises a cholesterol level.

8. The method according to claim 3,
wherein at least one of said plurality of biometric data values comprises a cholesterol level based on said measurement of the biosample.

9. The method according to claim 1, further comprising:
converting the first category health risk score to a first category health risk grade point average;
converting the second category health risk score to a second category health risk grade point average;
determining an overall health risk grade point average for the patient based on at least the first category health risk grade point average and the second category health risk grade point average; and
displaying the first category health risk grade point average, the second category health risk grade point average, and the overall health risk grade point average to the patient in real time on at least one of the electronic display and the printer.

10. The method according to claim 1, further comprising:
evaluating said first category health risk score for a plurality of patients to determine an aggregate first category health risk score;
converting said aggregate first category health risk score to an aggregate first category health risk letter grade;
evaluating said second category health risk score for said plurality of patients to determine an aggregate second category health risk score;
converting said aggregate second category health risk score to an aggregate second category health risk letter grade;
determining an aggregate overall health risk letter grade for the plurality of patients based on at least the aggregate first category health risk score and the aggregate second category health risk score; and
displaying the aggregate first category health risk letter grade, the aggregate second category health risk letter grade, and the aggregate overall health risk letter grade on at least one of the electronic display and the printer.

11. A system for generating and displaying a health risk assessment report to a patient in real time, comprising:
a personal computing device having a memory and a processor, the memory comprising computer-readable instructions which cause the personal computing device to receive a plurality of biometric data values for a patient; and
an electronic display operably connected to the personal computing device;
wherein the memory comprises computer-readable instructions which cause the personal computing device, using the processor, to automatically process a first subset of the plurality of biometric data values to determine a first category health risk score for the patient in a first health risk category and convert the first category health risk score to a first category health risk letter grade;
wherein the memory comprises computer-readable instructions which cause the personal computing device, using the processor, to automatically process a second subset of the plurality of biometric data values to determine a second category health risk score for the patient in a second health risk category and convert the second category health risk score to a second category health risk letter grade;
wherein the memory comprises computer-readable instructions which cause the personal computing device, using the processor, to determine an overall health risk letter grade for the patient based on at least the first category health risk score and the second category health risk score; and
wherein the memory comprises computer-readable instructions which cause the personal computing device, using the processor, to present the first category health risk letter grade, the second category health risk letter grade, and the overall health risk letter grade to the patient in real time using the electronic display.

12. The system according to claim 11, further comprising:
at least one diagnostic device capable of analyzing a biosample taken from the patient, said diagnostic device operatively connected to the personal computing device;
wherein at least one of the plurality of biometric data values is based on a measurement performed on the biosample using the diagnostic device.

13. The system according to claim 12, wherein said diagnostic device comprises a cholesterol meter.

14. The system according to claim 12, wherein said diagnostic device comprises a blood glucose meter.

15. The system according to claim 12,
wherein the biosample comprises blood.

16. The system according to claim 11,
wherein at least one of said plurality of biometric data values comprises a cholesterol level.

17. The system according to claim 12,
wherein at least one of said plurality of biometric data values comprises a cholesterol level based on said measurement of the biosample.

18. The system according to claim 11,
wherein the memory comprises computer-readable instructions which cause the personal computing device, using the processor, to present a simulated first category health risk letter grade and a simulated overall health risk letter grade to the patient in real time on the display; and
wherein the simulated first category health risk letter grade and the simulated overall health risk letter grade reflect a selected adjustment to at least one of the first subset of the biometric data values.

19. The system according to claim 11,
wherein the memory comprises computer-readable instructions which cause the personal computing device, using the processor, to evaluate said first category health risk score for a plurality of patients to determine an aggregate first category health risk score and convert said aggregate first category health risk score to an aggregate first category health risk letter grade;
wherein the memory comprises computer-readable instructions which cause the personal computing device, using the processor, to evaluate said second category health risk score for said plurality of patients to determine an aggregate second category health risk score and convert said aggregate second category health risk score to an aggregate second category health risk letter grade;
wherein the memory comprises computer-readable instructions which cause the personal computing device, using the processor, to determine an aggregate overall health risk letter grade for the plurality of patients based on at least the aggregate first category health risk score and the aggregate second category health risk score; and
wherein the memory comprises computer-readable instructions which cause the personal computing device, using the processor, to present the aggregate first category health risk letter grade, the second category health risk letter grade, and the overall health risk letter grade for the patient using the electronic display.

20. The system according to claim 11, further comprising:
a remote server for receiving said first and second category health risk scores from said personal computing device for a plurality of patients;
wherein the remote server is configured to evaluate said first category health risk score for the plurality of patients to determine an aggregate first category health risk score and convert said aggregate first category health risk score to an aggregate first category health risk letter grade;
wherein the remote server is configured to evaluate said second category health risk score for said plurality of patients to determine an aggregate second category health risk score and convert said aggregate second category health risk score to an aggregate second category health risk letter grade;
wherein the remote server is configured to determine an aggregate overall health risk letter grade for the plurality of patients based on at least the aggregate first category health risk score and the aggregate second category health risk score.
